(19) United States
(12) Patent Application Publication
(10) Pub. No.: US 2024/0009068 A1
(43) Pub. Date: Jan. 11, 2024

(54) TRANSFORMABLE PNEUMATIC SOFT STRUCTURES

(71) Applicant: Massachusetts Institute of Technology, Cambridge, MA (US)

(72) Inventor: Athina Papadopoulou, Concord, MA (US)

(21) Appl. No.: 18/041,918

(22) PCT Filed: Jul. 29, 2021
(86) PCT No.: PCT/US2021/043657

§ 371 Date: Feb. 16, 2023

Related U.S. Application Data
(60) Provisional application No. 63/066,752, filed on Aug. 17, 2020.

(51) Int. Cl. A61H 9/00

(52) U.S. Cl. CPC A61H 9/0092; A61H 2230/065; A61H 2230/425; A61H 2230/655; A61H 2201/5038

(57) ABSTRACT

A textile sheet having a thermoplastic coating on an interior of the chamber. The left and right-side area of the chamber are folded inward, towards each other, in a V shape, with the point of the V directed to the interior of the chamber. The textile sheet is heat sealed at ends of the sheet to render the chamber airtight, forming a 4-layer structure in at least lateral portions of each of a first and second end of the chamber with on the left side of the chamber, in order, a top portion of the textile sheet, an upper portion of the V shape, a lower sheet portion of the V shape, and a bottom portion of the textile sheet and with on the right side of the chamber, in order, a top portion of the textile sheet, an upper portion of the V shape, a lower sheet portion of the V shape, and a bottom portion of the textile sheet. The upper portion of the V shape being sealed to the top portion of the textile sheet in a plurality of locations along the chamber and the bottom portion of the textile sheet being sealed to the bottom portion of the textile sheet in a plurality of locations along the chamber.

### RELATED APPLICATIONS

This application claims priority to U.S. Provisional Patent Application No. 63/066,752, filed Aug. 17, 2020, the entire content of which is hereby incorporated by reference in its entirety.

### BACKGROUND

Haptic communication is a form of nonverbal communication, such as touch, that alters the medium in which humans and animals communicate. Humans relationships and emotional health may rely to a degree on contact and physical interactions with other humans. Haptic communication has therapeutic benefits via regulation of emotional states similar to deep pressure massage therapy or weighted blankets for promoting relaxation and reducing anxiety. Additional therapeutic benefits of haptic feedback include muscle rehabilitation and enhanced blood flow. Computer technology may be leveraged, with haptic communication techniques in mind, to enhance the sense of touch and motion, and mimic such emotions as love, and may be used to supplement emotional needs, such as for relationships and social interaction. Further, leveraging computer technology may be used to mimic positive haptic communication for individuals in times of stress, frustration, and social isolation.

### SUMMARY

A textile sheet having a thermoplastic coating on an interior of the chamber. The left and right-side area of the chamber are folded inward, towards each other, in a V shape, with the point of the V directed to the interior of the chamber. The textile sheet is heat sealed at ends of the sheet to render the chamber airtight, forming a 4-layer structure in at least lateral portions of each of a first and second end of the chamber with on the left side of the chamber, in order, a top portion of the textile sheet, an upper portion of the V shape, a lower sheet portion of the V shape, and a bottom portion of the textile sheet and with on the right side of the chamber, in order, a top portion of the textile sheet, an upper portion of the V shape, a lower sheet portion of the V shape, and a bottom portion of the textile sheet. The upper portion of the V shape being sealed to the top portion of the textile sheet in a plurality of locations along the chamber and the bottom portion of the textile sheet being sealed to the bottom portion of the textile sheet in a plurality of locations along the chamber.

A method that includes fitting an individual with at least one sleeve structure surrounding at least one body part of the individual, the sleeve structure comprising a plurality of pneumatic chambers, each of the pneumatic chambers wrapped around the body part, the pneumatic chambers disposed in a first determined order along a length of the sleeve structure. The method further includes coupling the pneumatic chambers to an actuator unit, the actuator unit operable under control of a processor and comprising at least one air pump, the actuator unit configured to inflate and deflate the pneumatic chambers in a determined sequence. The method further includes detecting a physiological pattern with sensors. The method further includes inflating and deflating, in the determined sequence, the plurality of pneumatic chambers disposed along the length of the sleeve structure.

### BRIEF DESCRIPTION OF THE FIGURES

FIG. 1. illustrates a sequence of folds for transforming a flat material sheet into a transformable pneumatic soft bar structure, according to embodiments.

FIG. 2. details a schematic of a transformable pneumatic soft bar structure before the stage of determining a negative curvature or positive curvature, according to embodiments.

FIGS. 3A and 3B illustrate transformable pneumatic soft bar structures with bonding patterns between folded layers for a resulting negative curvature shape, according to embodiments.

FIGS. 4A and 4B illustrate the transformable pneumatic soft bar structure of FIGS. 3A and 3B in its flat deflated state and in its curved state, after inflation illustrating transformation with negative orientation, according to embodiments.

FIGS. 5A and 5B illustrate transformable pneumatic soft bar structures with bonding patterns for positive curvature, with FIG. 5A representing the top part of a folded material and FIG. 5B representing the bottom part of a folded material, according to embodiments.

FIGS. 6A and 6B. illustrate the transformable pneumatic soft bar structure of FIGS. 5A and 5B in its flat state 6A and in its curved state 6B after inflation, inducing transformation with positive orientation, according to embodiments.

FIG. 7 illustrates a transformable pneumatic soft bar structure with alternate curvature (sinewave form), illustrating its flat state showing bonding patterns of the top side, according to an embodiment.

FIG. 8 illustrates a transformable pneumatic soft bar structure with alternate curvature (sinewave form), illustrating its flat state showing bonding patterns of the bottom side, according to an embodiment.

FIG. 9 illustrates the transformable pneumatic soft bar structure of FIGS. 7 and 8 with alternate curvature (sinewave form) in its curved (inflated) state, according to an embodiment.

FIG. 10. illustrates a transformable, pneumatic soft-surface structure fabricated from a single material sheet, according to an embodiment.

FIGS. 11A-C illustrate folding patterns for the transformable pneumatic structure of FIG. 10 to produce the assembled structure of FIG. 12, according to an embodiment.

FIG. 12 illustrates an assembled transformable structure fabricated from the single sheet of FIG. 10 using folding patterns according to FIG. 11A-C.

FIG. 13 illustrates multiple pneumatic soft bar structures of various sizes, that may connect together, resulting in a sleeve, according to an embodiment.

FIG. 14 illustrates that an assembled transformable structure may be fabricated having the multiple individual bars of FIG. 13, and that these bars may be of different sizes to taper structure with overlapping or non-overlapping bars to fit as a tubular structure or sleeve structure to fit a human limb, with each of the pneumatic chambers surrounding the human limb, the sleeve structures being disposed in a determined order along a length of the sleeve structure, according to an embodiment.

FIG. 15 is a photograph illustrating a sleeve transformable pneumatic soft bar structure having multiple bars and wrapped about a human limb, according to an embodiment.

FIG. 16 illustrates a transformable pneumatic soft bar structure with an incorporated heating pad aligned with a flat sheet and folded before end sealing, according to an embodiment.

FIGS. 17A, B illustrate a gripper made of transformable pneumatic soft bar structure in non-inflated state and inflated state, according to an embodiment.

FIGS. 18A-18F illustrate various garments that may be equipped with transformable pneumatic soft bar structures to provide haptic feedback or communications, or to provide treatment for medical or psychological issues, according to an embodiment.

FIG. 19A illustrates a person wearing the transformable pneumatic soft bar sleeve of FIGS. 13-15 to provide haptic feedback for psychological or psychiatric treatment, according to an embodiment.

FIG. 19B illustrates two people wearing the transformable pneumatic soft bar sleeve of FIGS. 13-15 to provide remote comforting capability to enhance relationships when the two people are unable to achieve physical contact, as further illustrated in FIG. 20B, according to an embodiment.

FIG. 20A illustrates that operation of the pneumatic soft bar sleeve may be initiated, or automatic operation adjusted, by remote operators during telemedicine sessions, according to an embodiment.

FIG. 20B illustrates that operation of the pneumatic soft bar sleeve may be controlled by sensors of a remote unit, according to an embodiment.

FIG. 21 is a block diagram of a computer system incorporating the haptic sleeve, according to an embodiment.

FIG. 22 illustrates timing for pressure of multiple pneumatic soft bar structures to be applied relative to the breathing cycle, according to an embodiment.

FIG. 23 is an illustration of limb compression including activation chambers of a pneumatic soft bar structure in sequence from proximal to distal along a limb, according to an embodiment.

### DETAILED DESCRIPTION

### Geometry, Fabrication & Transformation

### 1. Bar Structure

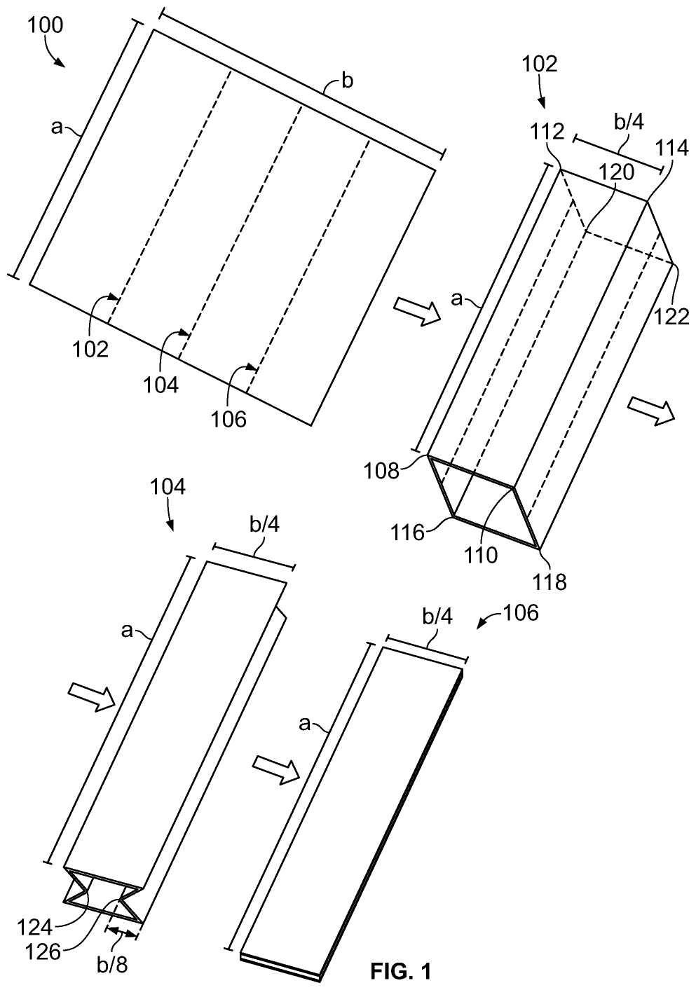

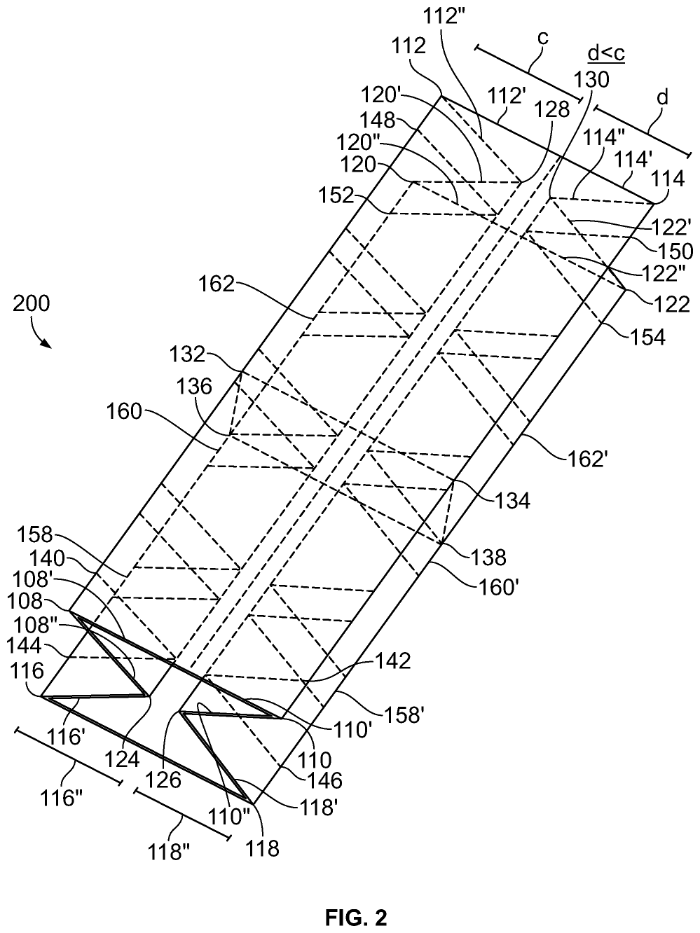

A bar structure can be formed from a single rectangular sheet of material 100 folded in a manner that creates a rectangular prism 101 (with its front and back face open), and then folded again in such manner that the side surfaces are folded inwards causing the top surface to fold onto the bottom surface, creating a two-level, four-layer structure, a pneumatic chamber 127. More precisely, a sheet of material 100 with dimensions a, b (a>=b) can be divided in 4 equal sections of dimensions a, b/4. The sheet of material 100 can then be folded along the lines 102, 104, 106 in a 90-degree angle parallel with the lines to form the rectangular prism 101. Then the face defined by the points 108, 110, 112, 114 is pushed towards the face of the points 116, 118, 120, 122, while at the same time the face defined by the points 108, 112, 116, 120 and the face defined by the points 110, 114, 118, 122 are folded inwards, towards each other, along their respective longitudinal axis (FIGS. 1 and 2), to form structure 123. The result being that the chamber formed has a top, a bottom, and two sides each with a point 124, 126 folded into an interior of the chamber. The result is a 4-layer structure in at least lateral portions of each of a first and second end of the pneumatic chamber 127 formed by the folded sheet with on the left side of the chamber, in order, a top portion of the textile sheet, an upper portion of the V shape, a lower sheet portion of the V shape, and a bottom portion of the textile sheet and with on the right side of the chamber, in order, a top portion of the textile sheet, an upper portion of the V shape, a lower sheet portion of the V shape, and a bottom portion of the textile sheet. The attaching together of the textile sheet at its ends renders the chamber airtight.

In embodiments, the upper portion of the V shape is heat sealed to the top portion of the textile sheet and the bottom portion of the textile sheet is heat sealed to the bottom portion of the textile sheet in a plurality of locations along the chamber. One of the following procedures is applied to the bar structure depending on the desired transformation on inflation:

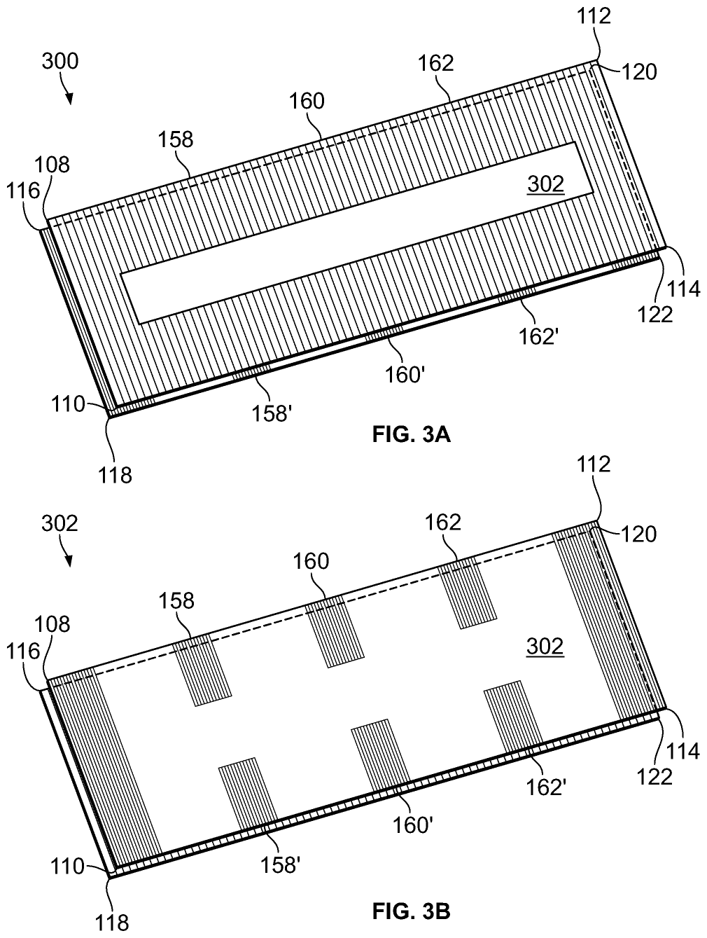

(a) Negative curve: The upper level is constrained in most of its area by having its two layers attached together (through heat sealing or other means) in most of their surface, except a thin strip in the middle to allow air from the top level to circulate into the bottom level (FIG. 3A). In an embodiment, this is done by heat sealing the upper portion of the V shape and the top portion of the sheet in many or all locations where they overlap in an uninflated state, while spacing well apart heat-seal locations attaching the upper portion of the V and top portion of the sheet. The bottom layer is constrained locally in at least two areas creating constrained strips along the structures' sectional axes (FIG. 3B). The strips take most of the length of the structure's short side (b/4, FIG. 1) except a thin strip in the middle to allow air to circulate throughout the structure's length.

The four layers, two of the top layers and two of the bottom layers, are attached together (through heat sealing or other means) in airtight manner at the two ends of the structure that are defined by its shorter sides. More precisely, in the embodiment of FIGS. 2, 3A, 3B, the upper layer of the top level defined by the points 108, 110, 112, 114 has three subdivisions, along its longitudinal direction as defined by the points 108, 108′, 110, 110′, 112, 112′, 114, 114′. Sides defined by points 108′ 110′ and 112′ 114′ define the thinner middle strip. Points 108″, 110″, 112″, 114″ are points on the sides defined by points 108⋅124, 110⋅126, 112⋅128, 114⋅130, respectively, and 116′, 118′, 120′, 122′, 116″, 118″, 120″, 122″, are points on the sides defined by points 116⋅124, 116⋅118, 118⋅126, 118⋅116, 120⋅128, 120⋅116, 122⋅130, 122⋅118, respectively, such as the lengths of each sides defined by the points 108⋅108′, 108⋅108″, 110⋅110′, 110⋅110″ . . . 122′⋅122″ are equal. The side defined by points 108⋅108′⋅112⋅112″ is bonded with side defined by points 116⋅116′⋅120⋅120′, and the side defined by points 110⋅110′⋅114⋅114′ is bonded with the side defined by points 110⋅110″⋅114⋅114″. Side defined by points 118⋅118′⋅122⋅122′ and the side defined by points 116⋅116′⋅120⋅120′ are bonded with the surface underneath them locally along the axes of the vertical subdivisions defined by points 158⋅158′, 160⋅160′. Sides defined by points 140⋅142 and 148⋅150 are axes on the top layer of the structure and the sides defined by points 144⋅146, 152⋅154 are axes on bottom layer of the structure. Areas defined by points 108⋅140⋅110⋅142 and 112⋅148⋅114⋅150 are each bonded with the surfaces immediately underneath them. Areas defined by points 116⋅144⋅118⋅146 and 152⋅120⋅154⋅122 are each bonded with the surfaces immediately above them. The result of the aforementioned method of heat-sealing various portions results in the pneumatic chambers 400, 402, as depicted in FIGS. 4A, B.

The plurality of locations along the chamber where the upper sheet is heat sealed to the upper portion of the V shape is more extensive than the plurality of locations where the lower sheet is heat sealed to the lower portion of the V shape such that upon inflation, the chamber curves towards the lower portion of the sheet

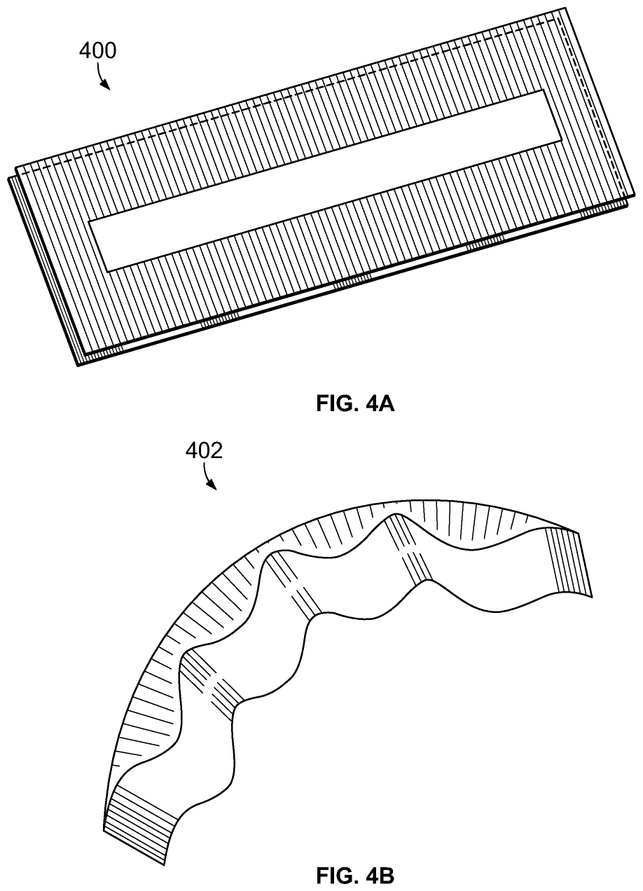

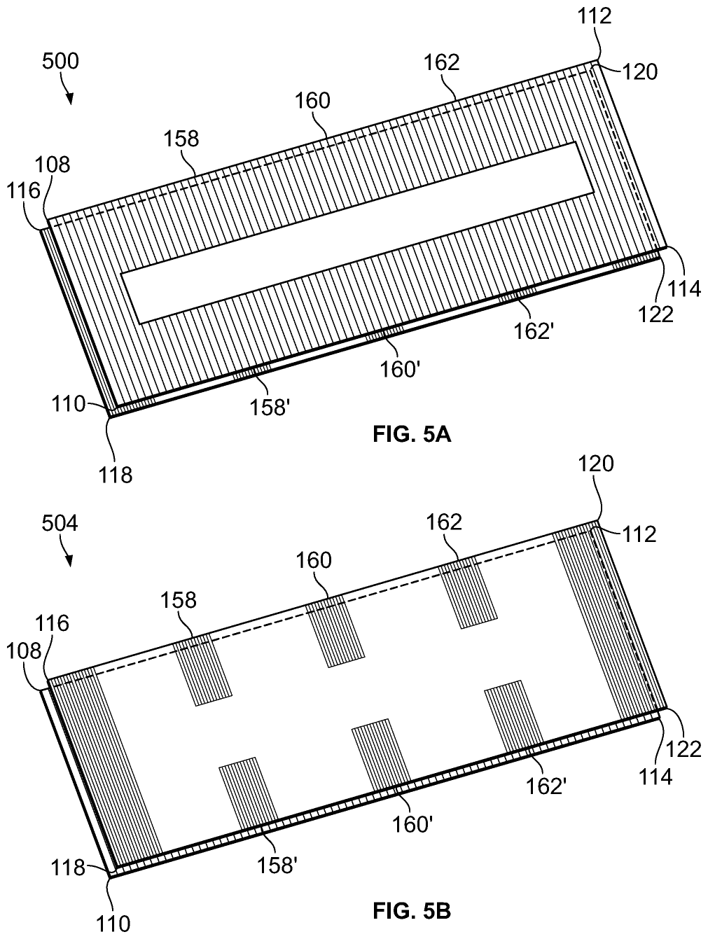

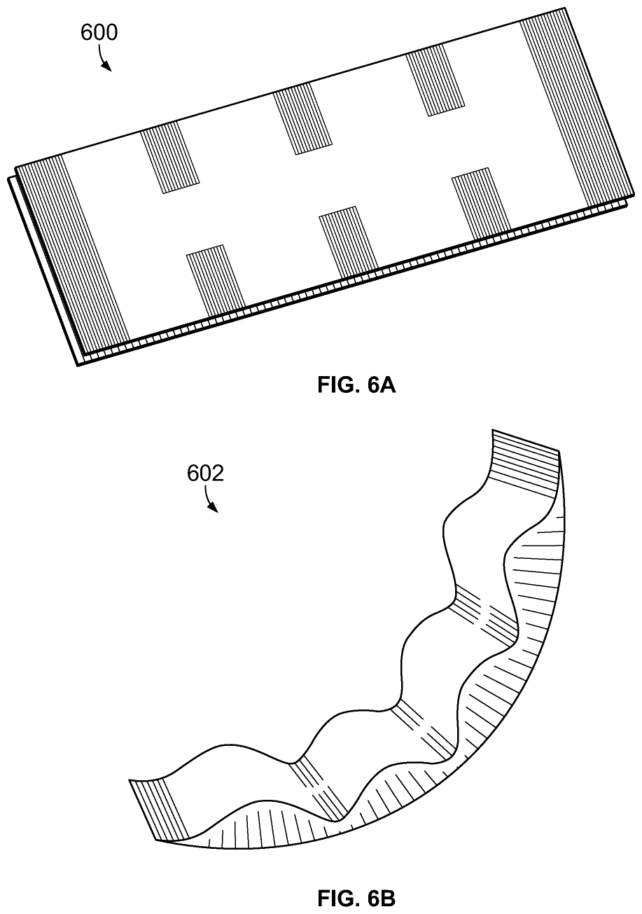

(b). Positive curve: A similar process is followed as described in the negative curve section but the procedure for the upper part of the structure is now applied to the lower part, and the procedure for the lower part of the structure is now applied to the upper part as illustrated in FIG. 2, FIG. 5A, 5B, FIG. 6A, and FIG. 6B. In an embodiment, this is done by heat sealing the lower portion of the V shape and the bottom portion of the sheet in many or all locations where they overlap in the uninflated state, while spacing well apart heat-seal locations attaching the upper portion of the V and top portion of the sheet. The uninflated state can be seen from both a top view 500 depicting the surface area defined by points 108⋅110⋅112⋅114 and a bottom view 504 defined by points 116⋅118⋅120⋅122. FIG. 6A shows the uninflated state 600 and FIG. 6B shows the inflated state 602.

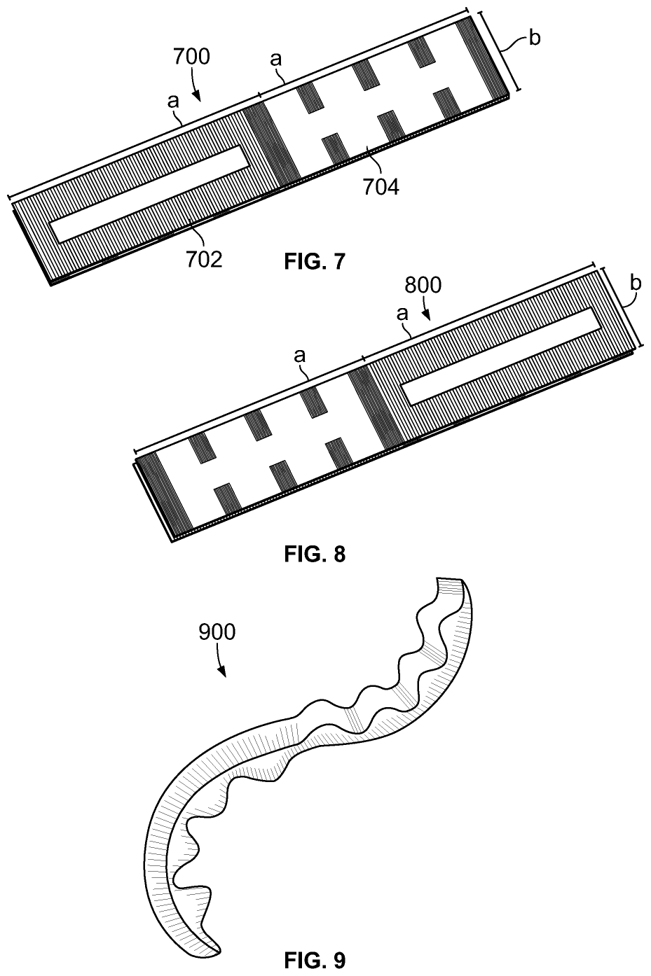

(c). Alternate curvature (sinewave form): A bar structure of alternate curvature 900 (FIG. 9) can be achieved if the procedures for the positive and negative curvature are applied to one or more parts of the bar structure in alternating manner. For example, if a bar 700, 800 (FIGS. 7, 8) with dimensions 2a (rather than dimension a, as described in the figures above), b is folded and bonded in such manner that half of the bar 702 (a part of dimensions a, b) is treated as a bar of negative curvature and the second part of the bar 704 (a part of dimensions a, b), is treated as a bar of positive curvature, then the bar will take on a sinewave form when inflated, as in the bar structure of alternate curvature 900 (FIG. 7, 8, 9). As another example, if on the bar described in section 1a (FIG. 2) there is a point 132 on the middle of the edge with points 108⋅112, a point 134 on the middle of the edge defined by points 110⋅114, a point 136 on the middle of the edge with points 116⋅120 and a point 138 on the middle of the edge defined by points 118⋅122, if the portion of the bar structure defined as the left part of the planar section defined by points 132⋅134⋅136⋅138 is being handled as a bar structure of positive curvature and the other half of the bar structure is being handled as a bar structure of negative curvature then we produce a bar structure of alternate curvature 900 (sinewave form).

The fabrication of the bar structures is fast and can be easily done manually with a handheld heat-sealing device, heat sealing industrial machine or custom tool, or by other bonding methods and materials that will ensure that the structure is airtight. In an alternative embodiment, although compartment ends are heat-sealed, the sealing of top and bottom V to top and bottom of the folded fabric sheet that results in curvature of each bar is performed with glue instead of heat-sealing. A combination of methods of assembly can also be used, such as sewing or glue, in the parts that do not need to be airtight. In the parts that are sealed through heating, sewing can be used as an additional procedure to add components like buttons or metal hooks. For structures made of fabric, the fabric needs to be purchased with one side coated with thermoplastics (for example nylon fabric coated with thermoplastic polyurethane (TPU)) or a thermoplastic coating can be part of the fabrication process. One of the advantages of the folding method is that only one side, the side on the interior of each bar, of the fabric needs to be coated.

### 2. Surface Structure Using a Single Material Sheet

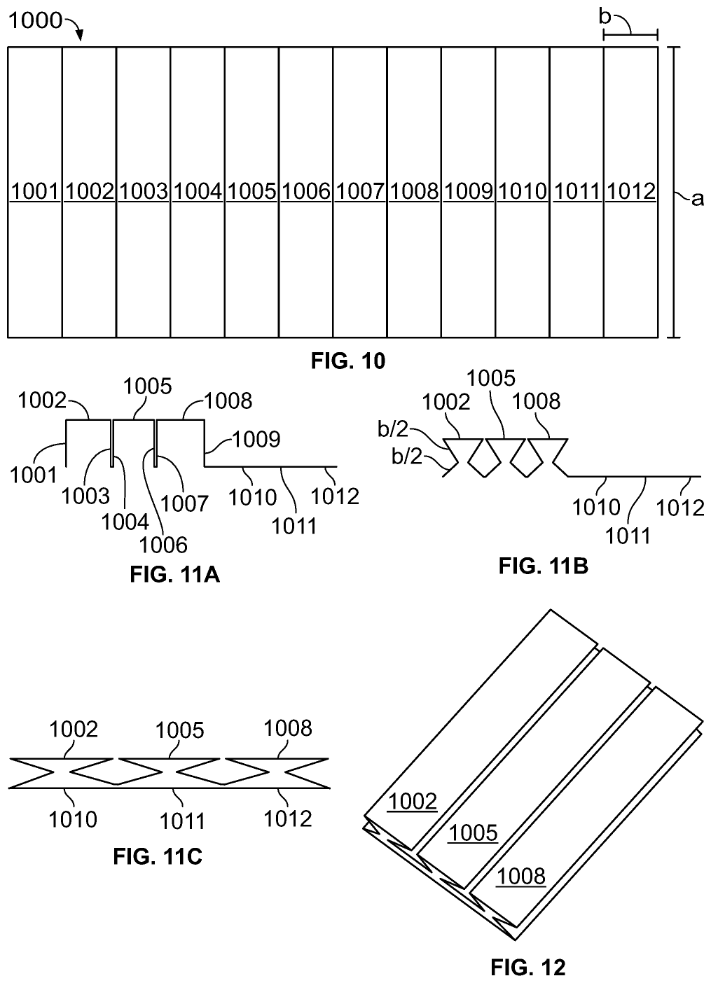

A surface structure 1000 can be achieved by folding a single material sheet and bonding together specific parts of it. The single material sheet has rectangular subdivisions—1001, 1002, 1003, 1004, 1005, 1006, 1007, 1008, 1009, 1010, 1011, 1012 shown as vertical subdivisions in FIG. 10.—each of dimensions a, b (b>=a). When the sheet is folded, parallel horizontal bar structures are created, in the following manner, as shown in FIG. 11: The first rectangular subdivision of dimensions a, b—subdivision 1001—is being folded in half along its longitudinal axis and the third rectangular subdivision—subdivision 1003—is being folded in half along its longitudinal axis in a manner that the two newly folded parts are positioned underneath the second rectangular subdivision—subdivision 1002. The same folding procedure is followed for the rectangular subdivisions 1004, 1005, 1006 and then for the rectangular subdivisions 1007, 1008, 1009 leading to a pleaded surface with three connected parallel bar structures formed by inflated subdivisions 1010, 1011, 1012 (with the bottom face open). Then, as shown in FIGS. 11B, 11C, and 12, material of the same sheet of length equal to 3b is being folded in a manner that covers all the bottom area of the 3-bar created structure. To prepare the fabrication of the structure in advance one needs to account for 4 times more material length than the desired length of the final bar surface structure (i.e. if the desired length of the surface structure is 3b, the material sheet should be 12b) (FIG. 10).

One of the following procedures may be applied to the bar structure depending on the desired transformation.

(a) Positive curvature: If we follow the steps described in the procedure of a positive curve of a single bar structure to the bar structures defined by the top surfaces of subdivisions 1002, 1005, 1008, and bottom surfaces of subdivisions 1012, 1011, 1010 the structure will transform from flat to curved when inflated, having a positive curvature.

(b) Negative curvature: If we follow the steps described in the procedure of a negative curve of a single bar structure to the bar structures defined by the top surfaces of subdivisions 1002, 1005, 1008, and bottom surfaces of subdivisions 1012, 1011, 1010 the structure will transform from flat to curved when inflated, having a negative curvature.

(c) Alternate (sinewave) curvature: An alternating positive and negative curvature (sinewave form) can be achieved if in the structure previously described the rectangular subdivisions 1001, 1002, 1003 . . . 1012 are each divided along their longer side (side a) in such manner that one part of the constructed bar structure is treated as a bar with positive curvature and another part of the bar structure is treated as a bar with negative curvature.

Double curvature: If a surface structure with length a, b (b>a) is divided in two equal parts in its longitudinal direction such as b1=b2, and the area a, b1 is treated as surface with positive curvature whereas the area a, b2 is treated as a surface with negative curvature, then a structure with double curvature is achieved.

Complex shapes and curvatures: More complex shapes and curvatures can be produced if the material sheet has unequal sides or elliptical subdivisions, resulting in a more complex surface (for example, an elliptical surface that when inflated transform into a cone).

### 3. Modular Structure Using Individual Bars

A modular surface structure using individual bars can be formed if multiple individual bar structures can be attached together and detached with minimal effort without affecting the bars' ability to be airtight and to transform in predefined shapes. For example, bar structure 1302 is a bar structure with four subdivisions 1301 in the bottom level of the structure. The areas of attachment of the two layers of the bottom level of the structure are indicated by the attachment areas 1304, 1306, 1308 and 1304′, 1306′, 1308′. Bar structure 1312 and bar structure 1322 are identical to bar structure 1302. The attachment areas 1314, 1316, 1318, 1314′, 1316′, 1318′, and 1324, 1326, 1328, 1324′, 1326′, 1328′ (1324′, 1326′, 1328′ are not shown because of over crowdedness) areas are equivalent to the attachment areas 1304, 1306, 1308, 1304′, 1306′, 1308′. Those attachment areas include buttons that can be pinched without affecting the ability of the structure to be airtight and its ability to transform in a predictable manner. Buttons can be sewed on these areas to allow temporary attachment between the individual bars: on each of the attachment areas 1304, 1306, 1308, 1314, 1316, 1318, 1324, 1326, 1328, a female snap button can be sewed, and on each of the attachment areas 1304′, 1306′, 1306′, 1314′, 1316′, 1318′, 1324′, 1326′, 1328′, a matching male snap button can be sewn so that each bar can snap on either of the other two. A surface can be produced if all three are snapped together (for example 1304 on 1314, 1306 on 1316, 1308 on 1318, 1314 on 1324, 1316 on 1326, 1318 on 1328) (FIGS. 10, 11A, 11B, 11C, 12).

### 4. Modular Wearable Sleeve Structure Using Individual Bars

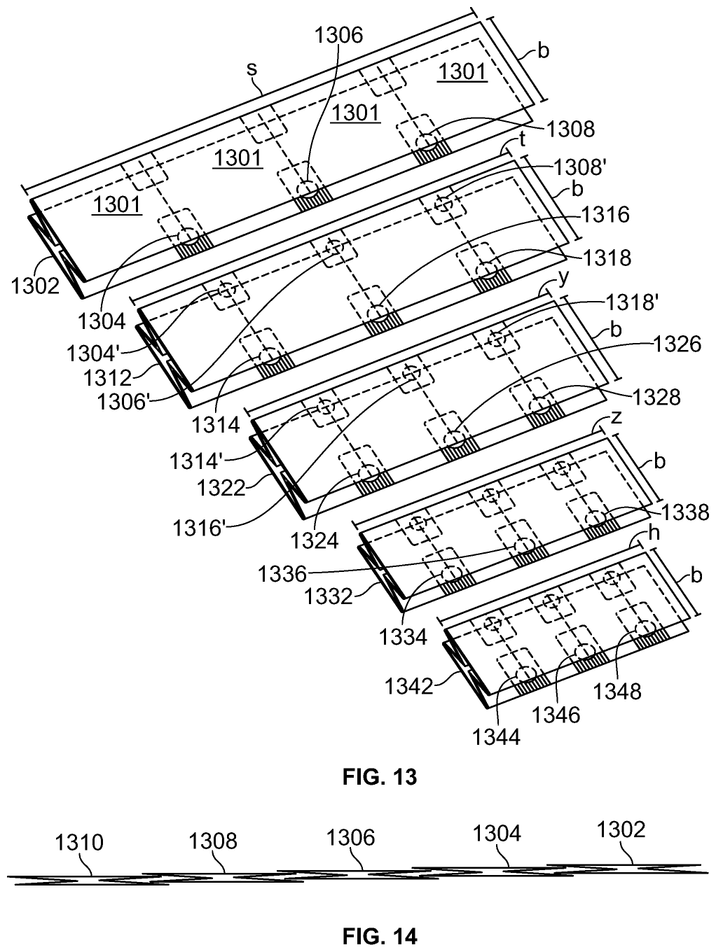

Bar structures 1302, 1312, 1322, 1332, 1342 have the same structure and number of divisions but vary in length such as the length of bar structure 1302, s<the length of 1312, t<the length of bar structure 1322, y<the length of bar structure 1332, z<the length of bar structure 1342, h in specific increment that does not exceed the length of the attachment areas where the snap buttons are located. Snap buttons are placed in the designated attachment areas in the manner that was previously described in section 3 so one side of each of the bars has female snap buttons and one side of each of the bars has male snap buttons. Sorting the bars by length, and snapping them together from the shortest to the longest, creates a surface of modular individual bars that can be worn as a sleeve that causes the feeling of pressure when inflated (FIGS. 13, 14, and 15).

### 5. Bar Structure Incorporating a Heating Pad

The making procedure of the bar structure as described in step one can be modified to incorporate a heating pad 1604. The structure 1600 can be made from a single material sheet of fabric coated 1602 on one side with thermoplastic material (TPU coated)—or other flexible inextensible material—with the added incorporation of a sheet of the same material and a heating pad 1604. The geometry, folding procedure and fabrication are described below. A single piece of coated fabric 1602 with dimensions a, b (a>=b) is placed with its coated side facing up. The sheet is divided into four equal rectangles, separated by lines 1606, 1608, 1610, of dimensions a, b/4. On top of one rectangle, an electric heating pad 1604 (or in alternative embodiments pads) is positioned centrally to its longitudinal axis. The fabric is pinched, at a location 1612 of the longitudinal axis, to allow for electrical wires to pass through. A second piece of coated fabric with its coated side on top is positioned centrally to the one rectangle's longitudinal axis covering the heat pad and the pinched hole at 1612. The space surrounding the second piece (dimensions surrounding the heating pad within the rectangle) should be sufficient to allow the rectangle to be attached to another surface in airtight manner through heat sealing. A hole at 1612 is pinched through the two material sheets on the central axis of rectangular A. A tube fitting is passed through the 1612 and secured in an airtight manner (FIG. 16).

The material sheet of dimensions a, b is then folded in such a manner that the rectangles are each rotated 90 degrees along the axes parallel to lines 1606, 1608, 1610. In the next step of the folding procedure, the face defined by the vertices 1622, 1624, 1626, 1628 is pushed vertically towards the face defined by the vertices 1630, 1632, 1634, 1636 while at the same time the face defined by the vertices 1622, 1626, 1630, 1634 and the face defined by the vertices 1624, 1628, 1632, 1636 are folded inwards along their respective longitudinal axis.

The desired transformation will dictate the orientation of the bar as described in the bar structure section. Utilizing bar structures with embedded heat pads in the wearable sleeve configuration as described in the previous section (section 4) can offer functional benefits regarding health and wellbeing applications as further discussed below (section 6).

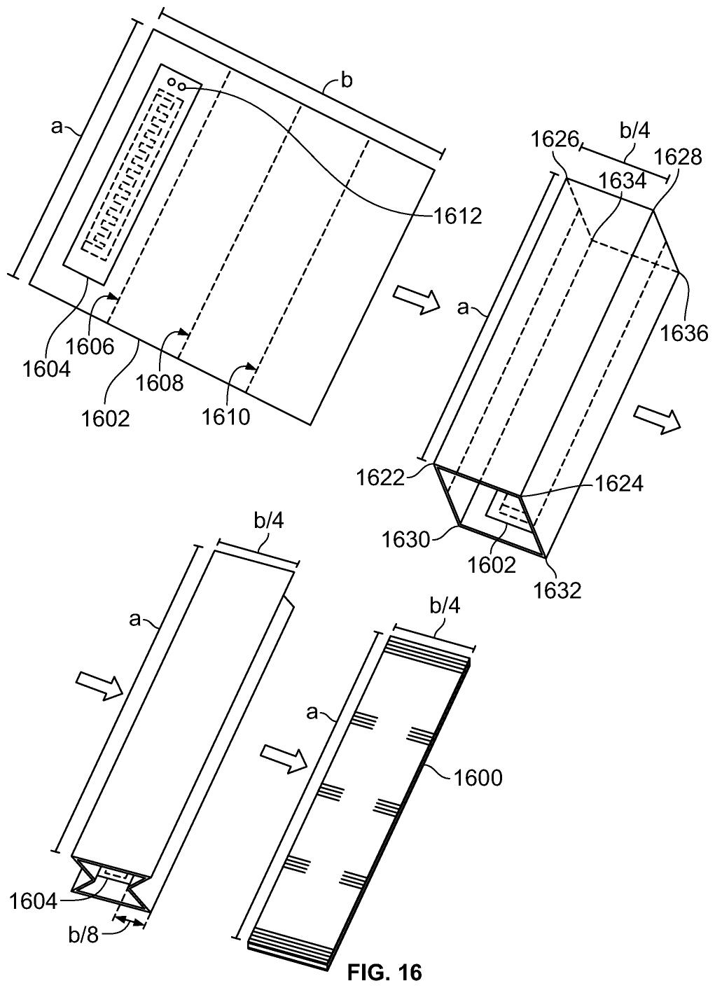

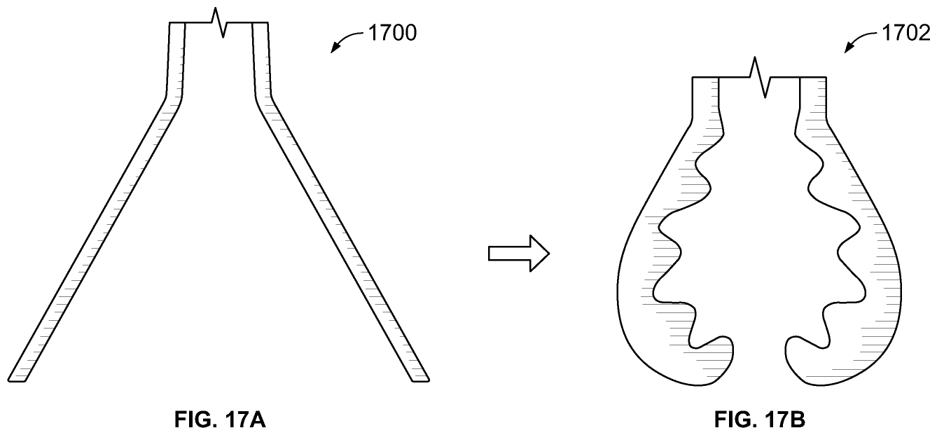

FIGS. 17A and 17B illustrate a gripper made of transformable pneumatic soft structure in non-inflated state 1700 and inflated state of the grippers 1702, as discussed herein.

### 6. Applications

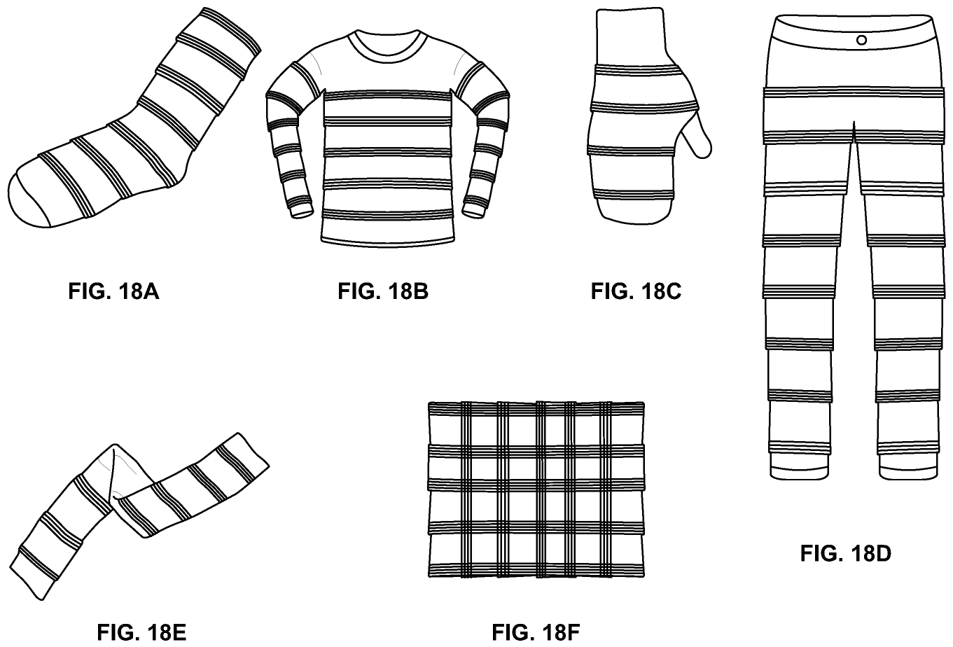

The transformable soft structures herein described may be fabricated into a variety of garments. For example, FIG. 18A illustrates a single haptic sock having multiple pneumatic bars of the transformable soft structures herein described, while FIG. 18B illustrates a jacket or sweater with two haptic sleeves along with chest and abdominal compression capability. FIG. 18C illustrates a glove equipped with multiple transformable soft structures of the type herein described. FIG. 18D illustrates pants with multiple transformable soft structures of the type herein described surrounding both legs and lower abdomen, 18E illustrates a scarf that may be wrapped around portions of a human body to provide squeezing feeling or a massage, and 18F illustrates a pillow that may provide massage to a human head or other body part resting on it.

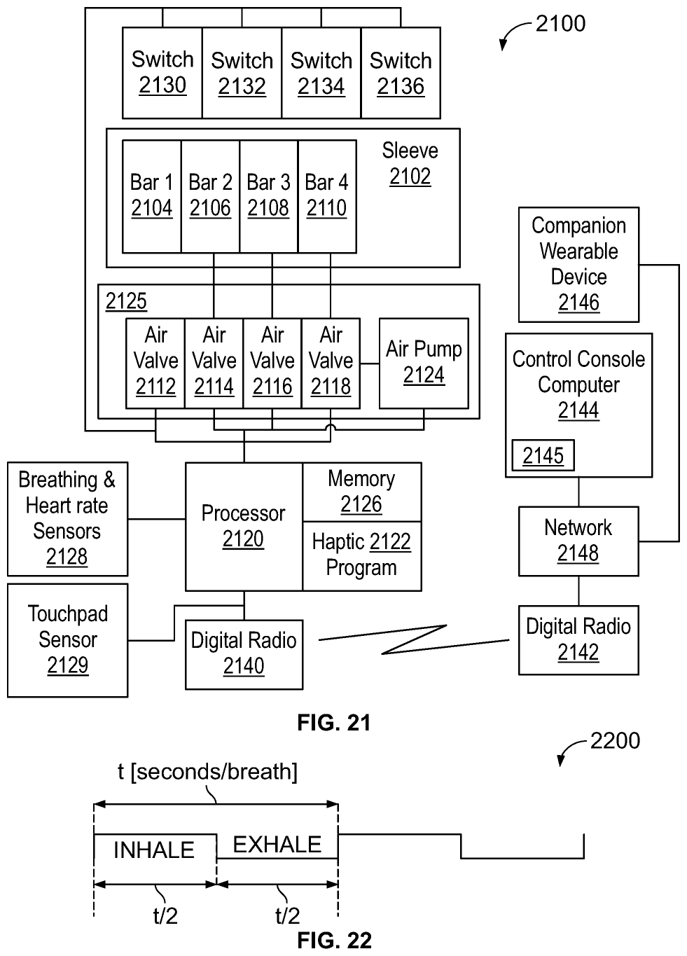

Embodiments of the present disclosure demonstrate an easy and affordable method of making transformable soft pneumatic structures (made of fabric or other flexible inextensible material) that can transform from a flat to a curved shape, in one or more directions. The method utilizes a system of folding and bonding patterns in single (continuous) sheets of flexible inextensible material such as nylon fabric. The transformation of the soft structure is caused by inflation of the structure through locations of connection to air pressure pumps or other mechanisms of gas inflation, described with reference to FIG. 21. For embodiments made of fabric, only one side of the fabric needs to be coated with thermoplastics (such as TPU) to allow for heat sealing. This allows for a variety of different fabrics to be used, making the invention suitable for a variety of applications. Another benefit of the invention is that the fabrication of the bar structure requires a simple hand-held, heat-sealing device, which is available in the market for low cost. In manufacturing processes automated heat-sealing machines can be used for faster production. Also, because a soft structure can be made from a single material sheet, the method reduces production costs by minimizing material waste and expediting the fabrication process.

Geometries composed of individual bar structures can be utilized as soft actuators and grippers (e.g., the grippers 1702, FIG. 17B) in robotic, manufacturing and engineering solutions. When the structure inflates, inflated pockets—surface bumps—are being created at the surface of the inner side of the structure that allow for more traction than a smooth surface. Inflatable soft structures can be used in aviation and automotive applications for safety or comfort solutions. As these structures easily change from a flat to a curved shape on demand, they can provide innovative solutions for interior architecture and furniture design allowing, for example, a mattress or pillow to take on an optimal shape upon inflation, and the bars may be of a smallest feasible dimension, such that they may transform to a shape that matches the contours of an individual's head (pillow, FIG. 18F) or body (mattress). The multilayer system of the structure allows for efficient resistance against physical loads making the invention suitable for seat or protective cushions. Transformable pneumatic soft structures can be used in the fashion and apparel industries for custom fit, and adjustable air insulation for warmth. In the fashion industry, fabric and garments can change shape on demand to address new market needs. In the shipping and manufacturing sector, the devices can be easily fabricated—just by folding single sheets made of coated fabric and heat sealing them. Transformable pneumatic soft structures can also be used in sports apparel for augmented performance through adjustable felt pressure caused by tunable inflation. Deployable architectural structures, transforming from flat to curved through inflation, can offer air insulation because of the air included in the structure, preserving interior heat levels, offering affordable and quick solutions in emergency environmental situations or nature sports when instant shelter is needed.

In the health sciences, transformable pneumatic soft structures can be used as a low-cost emergency solution in replacement of regular blood cuffs. Transformable pneumatic soft structures can be used in orthotic applications aiding in rehabilitation after limb injuries or neurological conditions causing limited stability and coordination. Transformable pneumatic structures can be used as exoskeletons for augmented performance or safety in environments of hazardous physical objects. The invention allows for seamless integration of heating pads that can augment their functionality as devices for enhancing wellbeing. In addition to the benefits of custom shape, fit, and pressure, transformable pneumatic soft structures can also provide the benefits of warmth—in applications such as warmth clothing and apparel, heated blankets, massage cushions, and medical heating wearable pads.

Transformable pneumatic soft structures can operate as massage devices aiding in better blood circulation; the embodiment of FIG. 18D may be used to reduce likelihood of blood clotting in human legs while patients are recovering from surgery or other procedures that require bed rest. Also, operating as wearable massage devices, transformable pneumatic soft structures, like those of FIGS. 18A, 18B, and 18F, can offer relaxation by lowering increased heart and breathing levels due to anxiety or stress.

### G-Suit and MAST Pant

The pant embodiment of FIG. 18D may also be used as a “G-suit” for pilots of high-performance aircraft, where inflation resists the tendency of blood to collect in the lower limbs while performing tight maneuvers in aircraft, or as “MAST-pants” for driving blood from legs into the body to help counter the effects of severe bleeding. In this embodiment, in addition to breathing rate and heart rate sensors, the system includes an accelerometer (e.g., accelerometer 2145).

### Transformable Structures for Psychological/Psychiatric Treatment and Communications Embodiments

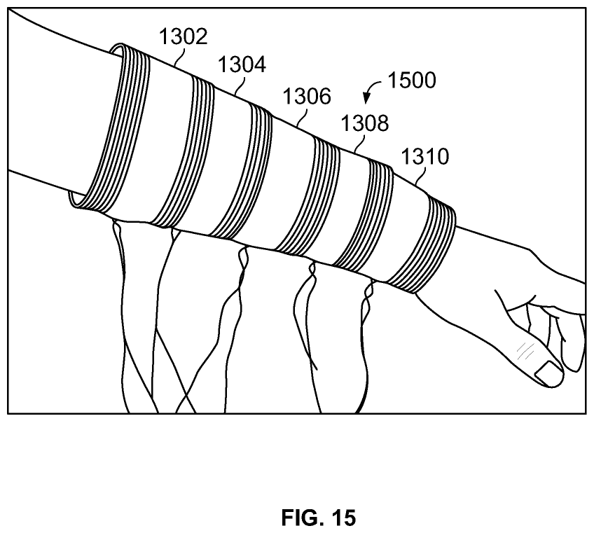

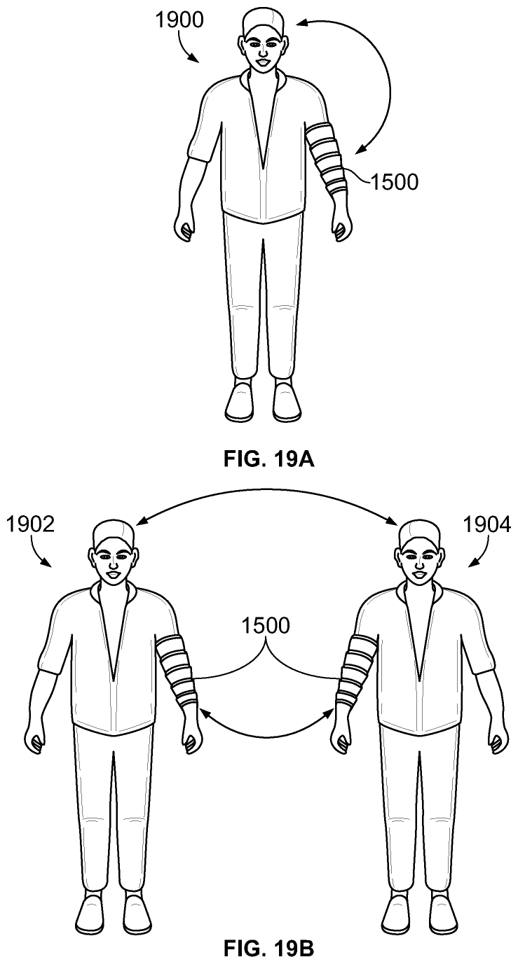

FIG. 19A illustrates a person 1900 wearing a sleeve 1500 (FIG. 15) to provide haptic feedback for psychological or psychiatric treatment through breathing regulation, or massage to improve blood circulation.

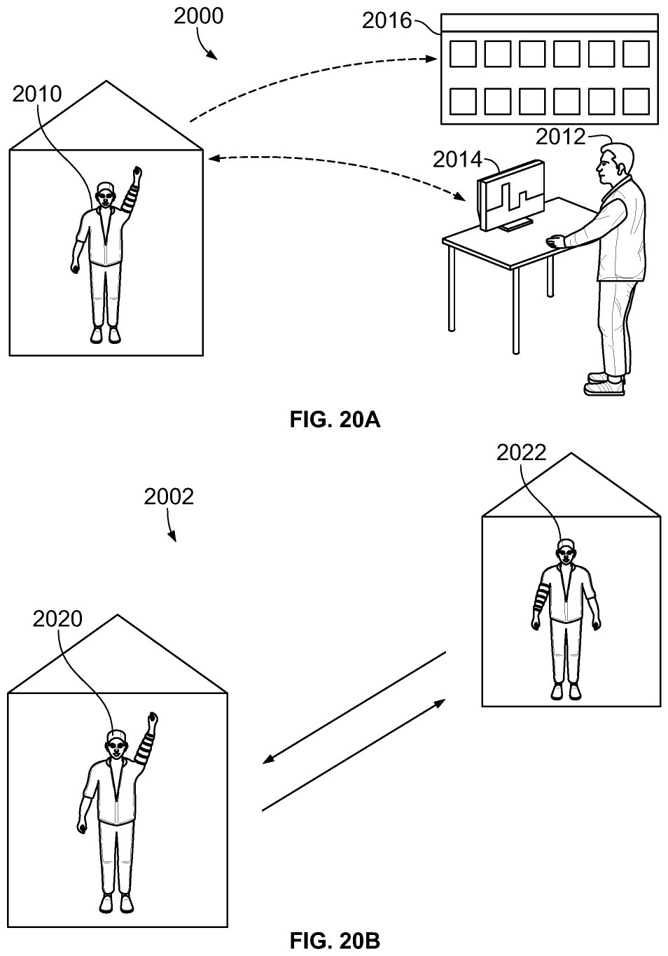

FIGS. 19B and 20B are described together and illustrate two people, a first person 1902, 2020 and a second person 1904, 2022 wearing sleeves 1500 (FIG. 15) to provide remote haptic communication to enhance relationships when the two people are unable to achieve physical contact. This embodiment uses the system 2100 discussed in further detail in FIG. 21. In this embodiment, the processor (e.g., processor 2120) of a first system (e.g., system 2100) communicates through the digital radio (e.g., digital radios 2140, 2142) and network (e.g., network 2148) with the second system (e.g., companion device 2146) and controls the inflation and deflation of the pneumatic chambers (e.g., bar structures 2104-2110) according to input on the touchpad of the second system, while transmitting commands entered on the touchpad to the second system.

In another embodiment, the processor of the first system communicates through the digital radio and network with the second system and controls the inflation and deflation of the pneumatic chambers of the first system worn by a first person (e.g., first person 1902, 2020) according to breathing, heartrate, or skin conductance (electrodermal activity) measured by the second system from a second person (e.g., 1904, 2022). In this way, the first person 1902, 2020 wearing the first system may cause the second system to inflate and/or deflate chambers of the second system, while the second person 1904, 2022 wearing the second system may cause the first system to inflate and/or deflate chambers of the first system. In the embodiment where the second system's breathing, heartrate, or skin conductance (electrodermal activity) sensors control inflation and deflation of the first system, the first person can experience an emotional state of the second person and vice versa.

Prior technological interventions utilizing rhythmic sensory stimuli, such as sound, vibration and light have demonstrated that rhythmic exposure to external stimuli can have an impact on our interoceptive awareness influencing the pace of our physiological functions (such as breathing, heart functions, and skin conductance). Interoceptive awareness is the awareness of the physiological state of our bodies and research has shown that is associated with our ability to express and process emotions. Lack of interoceptive awareness has been linked to various mental health disorders associated with the difficulty of processing or expressing emotions, including alexithymia, disassociation depersonalization, depression and psychopathy and developmental disorders such as autism.

The fact that external rhythmic sensory stimuli can help regulate our emotions can be linked to the phenomena of physiological synchrony and emotional contagion. Breathing techniques included in traditional meditation and yogic practices have long been considered as beneficial to health and wellbeing as they cultivate awareness of one's body promoting calmness.

The benefits of controlled slow breathing achieved through such traditional practices have also been demonstrated through technological interventions. Although mobile applications exist that provide auditory or visual feedback, the potential of applications with tactile feedback to regulate breathing, has only started to be explored.

Affective Sleeve, as Illustrated in FIGS. 13-15, for Promoting Calmness:

FIG. 21 shows a system 2100 composed from individual transformable pneumatic soft bar structures 2104, 2106, 2108, 2110, forming part of a wearable device in the form of a wearable device 2102, when they are attached together, or from a multi-bar sheet, as described above. Each bar structure 2104, 2106, 2108, 2110 is coupled to a separate electrically operable air exhaust and intake valve assemblies 2112, 2114, 2116, 2118 of an actuator unit 2125 operable under control of a processor 2120 of the actuator unit 2125. Exhaust and intake valve assemblies 2112, 2114, 2116, 2118 each retain pressure in associated bar structures 2104, 2106, 2108, 2110, vent air from the associated bars, or pass pressurized air from air-pressure pump 2124 to the associated bar structures 2104, 2106, 2108, 2110. In some embodiments, where bar structures 2104, 2106, 2108, 2110 have embedded electric resistive heat pads (e.g., heat pad 1604), switches 2130, 2132, 2134, 2136 are provided to selectively couple the resistive heaters of each bar to power. Particular embodiments may have fewer bars, such as two or three bars, or a single bar, or may have more bars, such as five or six bars or more. In a wearable device of FIGS. 13, 14, and 18 and as used in an ongoing study there are five bars each coupled, through a separate actuator unit 2125 with individually controllable inlet and vent valves, to air-pressure pump 2124.

Processor 2120 controls exhaust and intake valve assemblies 2112, 2114, 2116, 2118, air-pressure pump 2124, and switches 2130, 2132, 2134, 2136 under control of a haptic program 2122 in firmware in memory 2126 according to sensed breathing and heart rate as monitored by breathing, heartrate, or skin conductance (electrodermal activity) 2128 and operations indicated by input through touchpad sensor 2129 or communicated to it from a cellphone application (not shown). In embodiments, the haptic program 2122 controls inflation and deflation according to sensed breathing, heartrate, or skin conductance (electrodermal activity) according to modes set by a therapist or other authorized user (e.g., health-care professional 2012, FIG. 20A) at a computer console 2144. In other embodiments, the haptic program controls inflation or deflation according to inputs from a correspondent (e.g., the second person 2022, FIG. 20B) at a remotely located companion device 2146. In another embodiment, the processor 2120 of the system 2100 communicates through the digital radio 2142 and network 2148 with the second system and controls the inflation and deflation of the pneumatic chambers of the first system worn by a first person (e.g., the first person 2020, FIG. 20B) according to breathing, heartrate, or skin conductance (electrodermal activity) measured by the second system from a second person.

The processor 2120 is thus coupled through the exhaust and intake valve assemblies 2112, 2114, 2116, 2118 to control inflation and deflation of the bars structures 2104, 2106, 2108, 2110 (pneumatic chambers). The wearable device 2102 is portable and is operated from a rechargeable battery (not shown). The wearable device 2102 includes a digital radio 2142 coupled to processor 2120 for linking to, and wirelessly communicating through, a digital radio 2142 and network 2148 to and from computer console 2144 or remotely located companion device 2146. In a particular embodiment, the digital radio 2142 is IEEE 802.11 (WiFi) compatible, and in another embodiment is compatible with Bluetooth radios.

The wearable device 2102 can provide tactile feedback in the form of pressure by inflating and deflating in rhythmic manner and in the form of warmth when heat-pads are integrated in the device. Feedback in the form of tactile sensations has been proven to have therapeutic effects on health. Particularly the sensation of pressure in the context of massage therapy has been proven to have therapeutic effects on emotional health. The inclusion of warmth reinforces the health benefits and therapeutic effects of the device as research demonstrates the significant positive effects of warmth in lowering stress levels.

When the sleeve is composed of soft heat-sealable fabric bar structures 2104, 2106, 2108, 2110 (as in the case of the pneumatic soft structures) each cuff can operate solely as a pneumatic cuff or can have a heat-pad (e.g., heat pad 1604) integrated seamlessly as described in the disclosure. For the function of the sleeve (e.g., wearable device 2102), a system (e.g., system 2100) for pneumatic and temperature control is required (if heat sensation is also produced). For optimized pneumatic control each bar cuff is connected through an air tube to an electrically operable pneumatic valve and vent assembly (e.g., exhaust and intake valve assemblies 2112, 2114, 2116, 2118, any of which may take the form of a solenoid valve) which is connected to an air-pressure pump 2124. This allows for autonomy in the behavior of each individual cuff. For even greater autonomy each cuff can be connected to its own mini air pump. The pneumatic switches and pump (or pumps) are connected to a series of switches (e.g., switches 2130, 2132, 2134, 2136) connected to a controller that is programmed to determine the connections to the incoming current depending on the desired behavior of haptic action.

To be effective, the haptic action of the affective sleeve device (e.g., wearable device 2102) is correlated to the wearer's rhythmic physiological functions, such as breathing, heart rate, and skin conductance (electrodermal activity). Studies related to the present disclosure have focused on the correlation to breathing, the impact of the device on the wearer's breathing rate, and subsequently their subjective affective state. Prior studies have demonstrated that providing false physiological feedback can regulate the heart or breathing rates. The therapeutic method of the affective sleeve builds upon evidence of the impact of false physiological feedback (that is, exposure to external stimuli in rhythmic patterns correlated to physiological functions but with different rates than the actual function—thus false feedback). In short, the physiological impact of the sleeve lies in the fact that the wearer's breathing tends to synchronize with the sleeve's rhythm of haptic action. In addition, skin conductance (electrodermal activity) measurement determines levels of physiological arousal and may be used to determine stress levels and positive excitement. Skin conductance does not provide a rhythmic pattern as in breathing or heart rate but a signal may be measured, which includes peaks and valleys depending on one's startling response or overall stress levels.

### Haptic Action Cycle in Response to Breathing

The haptic action of the sleeve mimics the breathing cycle consisting of inspiration and expiration as in FIG. 22. If we were to match the haptic action to the actual breathing rate of the wearer and if a wearer's breathing rate is equal to 16 bpm, then the sleeve would also have 16 full cycles per minute of activation/deactivation. In the case of a pneumatic sleeve without integrated heat-pads, the activation/deactivation cycle consists of inflation (pressurization) and deflation (depressurization) with the sleeve's inflation synchronizing with the wearer's inspiration and the sleeve's deflation presumably synchronizing with the wearer's expiration, or vice-versa. In the case of a pneumatic sleeve with heat-pads integrated and activated, the activation cycle consists of inflation and warmth increase, simultaneously, and the deactivation cycle with deflation with warmth decrease, simultaneously.

It is hypothesized (with positive evidence from the studies discussed below) that haptic action with a cycle shorter than the wearer's full breathing cycle causes the wearer's breathing rate to increase. Conversely, it is hypothesized that a haptic action with inflation/deflation cycle longer than the wearer's breathing activity cause the wearer's breathing rate to decrease.

### Sleeve Activation Patterns and Parameters

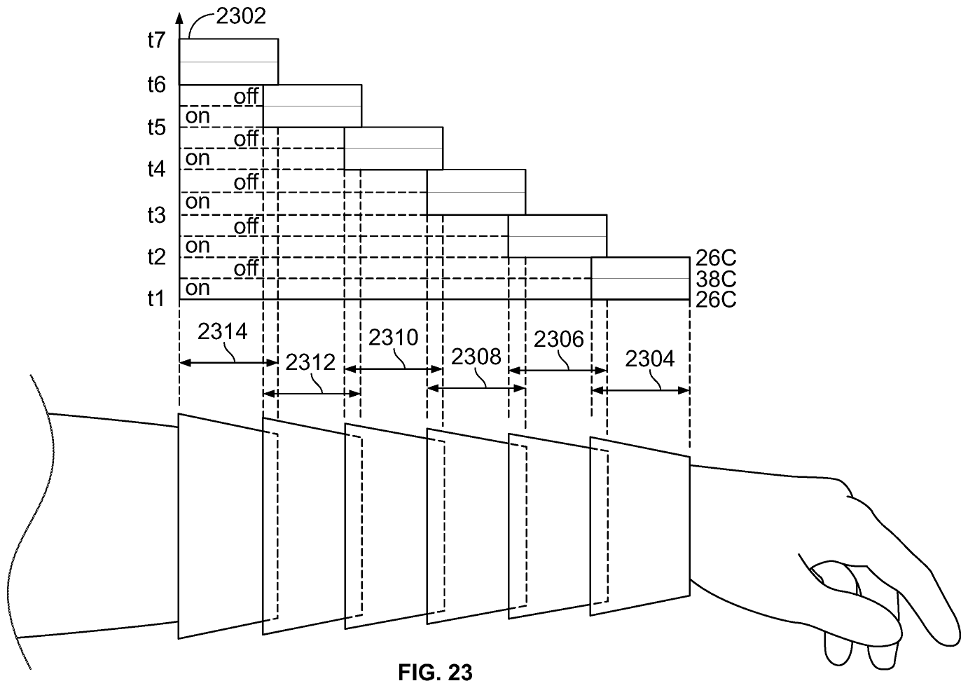

Different activation patterns of haptic action can be implemented activating the wearable device's 2102 bar structures 2104-2110 in sequence or simultaneously in periodic manner with an on/off cycle equal, greater, or lower to the wearer's relaxed breathing cycle. The sequence can have one direction as illustrated in FIG. 23. For example, as indicated by the graph 2302 showing time as a function of certain bars activating, it can be seen that bar 2304 is activated first, at time t₁, followed by bar 2306, at time t₂, followed by bar 2308, 2310, 2312, 2314, at times t₃, t₄, t₅, t₆, respectively. Likewise, the direction of bars activating can be bidirectional switching directions in sequential, random, or another programmed manner. For example, an activation pattern of sequential haptic action can have a direction from the wrist to the elbow, as shown in FIG. 23, or a sequence from the elbow to the wrist or a sequence from the wrist to the elbow and back to the wrist.

Pressure intensity: The amount of pressure during the activation sequence can vary in location (per cuff) or time or remain consistent (when in full inflation). The pressure could be light enough to be subliminal or stronger but should not exceed the felt pressure of a blood pressure cuff.

Warmth intensity: Warmth can be produced at the same level in all cuffs when activated or can vary in intensity per cuff. The warmth sensation could be lighter or stronger and may be in line with the warmth felt by a common commercial heat-pad.

Cycle of haptic action: The cycle of haptic action can be longer or shorter than the wearer's actual breathing cycle in consistent manner throughout the sequence or vary in length during the sequence in certain programmed manner.

Sensors/Input Signal from Physiological Functions

To provide haptic action in response to physiological functions, the sleeve device is programmed based on input from physiology sensors. The device can receive input in real-time, updating its cycle of haptic action based on the physiological state of the wearer. In this case, the haptic action of the device takes the form of continuous haptic feedback as it updates its response based on changing physiology input. Although this setup is in principle more effective, a simpler setup can also be effective: the sleeve can be programmed based on measured physiology signals when the wearer is in relaxed state to provide custom haptic-action based on this one-time feedback system.

Physiology sensors measuring heart activity (pulse) and skin conductance (i.e., electrodermal activity), can be integrated in the sleeve (e.g., wearable device 2102) as these measurements can be retrieved from sensors located on the wrist. Physiology sensors measuring breathing activity need to be placed on the chest, and therefore cannot be integrated in the sleeve, unless the sleeve is part of a custom garment, such as a shirt or suit. The sensors do not necessarily need to be integrated in the device for it to function or be effective if the device is connected to sensors or preprogrammed based on the wearer's physiology input.

The sensors can be physically connected or the input can be received wirelessly through a network communication system. In the case of a network communication system, both the sleeve device and sensors need to be connected to a central server to achieve continuous feedback. A network system can also facilitate remote monitoring of the sleeve device from a medical professional, health-care provider, or mental health professional, as discussed with reference to FIGS. 20 and 21.

### Testing and Results

### Study 1

Eighteen students were recruited to participate in a study that required them to wear a sleeve with haptic action while taking a quiz to induce stress. The sleeve was constructed differently than the pneumatic sleeve previously described, in that the sleeve used in this study had electrically heatable wires of a shape memory alloy that would compress the participant's arms to provide sensations of warmth and pressure when the wires of shape memory alloy were heated. The sensations of pressure provided by this shape-memory-alloy sleeve resemble those provided by the pneumatic sleeve herein described. The electrically heated wires were controlled by a processor programmed to provide haptic action including warmth and pressure in sequence, activating one wire after the other, in direction from the wrist to the elbow (FIG. 23).

The sleeve was programmed to provide haptic action in two modes:
(1) Slow (where a full cycle of haptic action is equal in duration to the wearer's full breathing cycle at relaxed state);
(2) Fast (where a full cycle of haptic action is 25% shorter than the wearer's breathing cycle at relaxed state); Participants were randomly assigned to a group of Slow haptic action, Fast haptic, or the Control group (where the participants wore a sleeve but the sleeve remained inactive).

The tested hypothesis was that slow and fast haptic action of the affective sleeve could decrease and increase, respectively, participants' psychophysiological symptoms of stress. Throughout the study participants were wearing a sensor on the chest to collect data related to their breathing activity (BR) and a sensor on their wrist to collect data relate to their electrodermal activity (EDA). Data were gathered from baseline measurements prior to the study and the sleeve was customized to produce haptic action relative to the wearer's BR mean-baseline measurement value.

A comparison of the mean change in BR from relaxed to stressed conditions showed that the group with slow haptic action had a smaller increase in breathing rate than the group with fast haptic action. Comparisons between the Control and Fast groups show that breathing rate was significantly higher in the Fast group. These results suggest that a faster pace of haptic action along the sleeve may increase the rate of respiration. Results from the EDA data showed that the mean change in EDA from relaxed to stressed conditions was lower in the Slow group than in the Fast and Control groups, however, with no statistical significance. Results from the self-reported data showed a positive correlation between perceived calmness and warmth, and negative correlation between perceived calmness and the speed of the haptic action. Most participants reported that slow haptic action had a calming effect.

### Study 2

Study 2 uses a pneumatic sleeve having heat-sealed, folded, compartments driven by electrically-operated valves from an air pump, as herein described. The pneumatic sleeve used has a modular design and is comprised of five individual cuffs each with an integrated heat-pad to have the capacity to also provide warmth as part of its haptic action. The goal of this study is to evaluate different activation patterns on the wearer's physiology and affective state. The pneumatic affective sleeve has the advantage that warmth and pressure stimuli can be produced simultaneously or separately as the production of one is not tied to the production of the other (as in the case of an alternative embodiment using a sleeve made from shape memory alloys). The pneumatic sleeve has also the advantage of being more robust and reliable in its actuation than the one made of the shape memory alloys.

In this study, there is no stressing condition as part of the procedure; the participants are experiencing seven different conditions of haptic action involving different activation patterns. The conditions are experienced in random order. The participants are asked to evaluate the affective impact of the haptic action before and after each condition. The device is wirelessly connected to a central server which is also connected to a user interface including study instructions and the user surveys. A sensor placed on the wearer's chest is measuring the wearer's breathing rate throughout the study and a sensor placed on the wearer's fingers is measuring the wearer's electrodermal activity throughout the study. Baseline measurements are also taken prior to the study.

The study tests the impact of different paces of haptic action to participants' psychophysiological state under the hypothesis that the higher the pace of haptic action compared to the wearer's relaxed pace will lead to a greater increase of the wearer's BR and the lower the pace of haptic action compared to wearer's relaxed pace will lead to a decrease in the wearer's BR. Secondly, the study aims to investigate whether the different activation patterns and types of haptic action (pressure only or warmth and pressure) have a different psychophysiological impact to the wearers—particularly to test the hypothesis that each condition is correlated with a distinct affective state.

To test these two hypotheses the participants are assigned to one of the following three groups: “Slow” (where the haptic action cycle is 30% longer than the wearer's BR cycle in relaxed state), “Regular” (whether the haptic action cycle is equal to the wearer's BR cycle in relaxed state), and “Fast” (whether the haptic action cycle is 30% shorter than the wearer's BR cycle in relaxed state). The same seven different conditions are being tested in each group in random order. The conditions are the following: (1) sequential activation in direction from the wrist to the elbow, in full haptic-action cycles, with haptic action consisting only of pressure; (2) sequential activation in direction form the wrist to the elbow, in full haptic-action cycles, with haptic action consisting of pressure and warmth; (3) simultaneous activation of all cuffs, in full haptic-action cycles, with haptic action consisting only of pressure; (4) simultaneous activation of all cuffs, in full haptic action cycle, with haptic action consisting of pressure and warmth; (5) sequential activation in direction form the wrist to the elbow, in half haptic-action cycles (equal to half breathing cycle), with haptic action consisting only of pressure; (6) sequential activation in direction form the wrist to the elbow, in half haptic-action cycles, with haptic action consisting of pressure and warmth; (7) no haptic action—the sleeve remains inactive.

The results so far are aligned with the hypothesis that the higher the pace of haptic action, the higher the breathing rate; and the lower the pace the haptic action, the lower the breathing rate. Based on the analysis of mean BR change from baseline to study measurements, the Fast group had a higher BR than the baseline (increase from baseline), the Regular and Slow group had a lower BR than the baseline (decrease from baseline) with the Slow group having greater decrease compared to the Regular group. The haptic action including warmth intensifies the calming effect.

1. Therapeutic Function of the Sleeve with Haptic Action

A sleeve that can effectively regulate breathing rate to promote calmness can be useful to people suffering from anxiety disorders. If the sleeve functions on a real-time continuous physiology input, it can serve as a biofeedback device to allow one to self-regulate in a noninvasive manner. The device can be programmed to provide a constant relaxed haptic action pace or can be programmed to trigger its action once a high rate of physiological functions is being detected. Other physiological measurements can also be considered to determine whether the changes of physiological functions are due to intense physical activity or due to mental distress.

Individuals with sensory processing disorders—which are associated with a mental health disorder or a development disorder—utilize special products available on the market such as heavy blankets or squeezable jackets that provide comfort and alleviate the distress caused by the sensory triggers. However, responses to the environment and distress triggers highly differ from individual to individual with such disorders. This mean that there is a need for customized solutions that can be programmed according to individual needs. A pneumatic sleeve with haptic action can be programmed to address such needs.

When the signals received by the physiology sensors are transmitted wirelessly, the system provides the opportunity for remote monitoring by a health-care professional 2012 (FIG. 20A). This can allow the professional to enable certain modes or functions of the sleeve based on the detected physiology levels. Remote mental healthcare and telemedicine is usually limited to consultations and prescriptions. However, monitoring the physiological expression of emotional distress can open another pathway for non-invasive wellbeing solutions. More important, individuals can self-monitor themselves and gain greater awareness of the own phycological and physiological reactions.

Telemedicine with the use of the sleeve with haptic action can be achieved when the patient 2010 is wearing the sleeve, in communication with a remote location 2016, and the health-care professional 2012 is monitoring the sleeve remotely through a computer console 2014, such as console computer, or mobile interface receiving real-time updates of the patient's physiology signals. Information access to the sleeve can happen in two ways: (1) the monitoring health-care professional 2012 (FIG. 20A) can receive the information in visual form on a computer console 2014 as an output of the signal or encoded in a message (2) both the monitored and monitoring individual are wearing a sleeve. In the latter case, the monitoring individual feels the physical output of the emotional state of the monitored individual directly in the form of haptic action (warmth and pressure) in their own sleeve (FIG. 20B). A therapeutic feedback loop can then be created between the monitored and monitoring individuals.

In a particular embodiment, one or more haptic sleeves of a garment may be inflated or deflated by the processor according to physiological stress detected by the processor through the breathing and heart rate sensors according to program parameters set by a therapist, or another authorized user, through computer console 2014, 2144. The wearable form of the pneumatic soft structures is not restricted to a sleeve. A whole jacket, or even a body suit can be fabricated to operate in a broader area of the body FIG. 18B). In that case the activation pattern can be sequential starting for example from the wrist of one arm and ending at the wrist of the other arm after having been activated at the core of the body.

Pneumatic soft structures with haptic action can have an impact on our psychophysiology because they are in contact with our body. This fact opens the pathway for the design of different kind of objects beyond wearables and garments. A pillow, a scarf, an inflatable armchair or mattress, are some of the possible objects that can made to provide haptic action based on the same principles of emotion regulation though breathing (FIG. 18A-18F).

Communication through a sleeve or other objects with haptic action does not need to be limited to therapy or health monitoring. Such objects can help establish remote haptic communication between socially or emotionally connected individuals (FIG. 20B). Such haptic mode of remote communication can allow emotion exchange in non-verbal means by allowing one individual to directly feel how the other individual feels without the need of verbal communication. This is particularly useful in the case of individual with limited verbal capacity as in nonverbal autism.

### 2. Physical Health: Lymphatic System and Blood Circulation

Since ancient times, patients have benefited from the positive effects of haptic therapeutic treatment including massage therapy and other systematic forms of applied pressure. Practices involving physical therapy are usually considered essential part of any physical rehabilitation process leading to the recovery of muscle, joint injury, surgery or illness. Haptic treatments with systematic applied pressure significantly enhance blood circulation and stimulate the lymphatic system, contributing in the treatment of the various venous and lymphatic system. The lymphatic system is a network of tissues and organs that aid the body in releasing toxins and fighting against infections by transporting lymph, a fluid containing white blood cells, throughout the body.

When there is a blockage in the lymphatic system, one can suffer from a condition called lymphedema. The blockage can be created by either a malformation or dysplasia of the lymph vessels and/or nodes, lymph node removal or damage to the lymphatic system due to cancer treatment, infection, obesity and other causes. The blockage leads to lymph fluid build-up in the fatty tissues under the skin because the lymphatic system is unable to properly move the fluid back to the blood stream. This causes swelling to affected parts of the body, usually the arms or legs. The swelling causes pain and discomfort reducing the quality of life of the individual suffering from the condition, and when severe can cause serious life-threatening health complications.

The first-line treatment of lymphedema is a specialized physical therapy, called Complete Decongestive Therapy (CDT) which includes manual lymphatic drainage through massage, exercise and maintenance management using compression stockings and/or intermittent pneumatic compression pumps, to aid in the removal of the concentrated fluid of the tissue and enhance circulation. Intermittent pneumatic compression pumps are devices consisting of a pump and an inflatable sleeve or garment comprised of multiple pneumatic chambers that inflate sequentially, one after the other in the necessary direction to aid in the lymph fluid drainage and blood circulation. Such pumps are sometimes used as stand-alone treatment or more often as a complement to the physical manual treatment. Depending on the machine used, throughout the compression treatment the patient must remain seated or lying down. Such devices are not designed to be mobile; they are usually heavy and bulky constraining body movements. The devices are also not affordable as they usually cost thousands of dollars. Devices may be available to rent or to use in special health-care centers.

Contrary to the intermittent pneumatic compression pumps available in the market, a compression device made from the pneumatic soft structures can be affordable and lightweight, allowing full mobility. Maximum mobility is ensured because the pneumatic soft structures when inflate do not increase their volume outwards but only inwards—towards the skin. Medical suits, jackets, sleeves or pants can easily be fabricated from pneumatic soft structures aiding in medical treatment on-the-go, all day long promoting optimum blood flow and proper function of the lymphatic system.

Intermitted pneumatic compression is also used as a therapeutic technique to improve venous circulation in patients who suffer from edema, risk of deep vein thrombosis (DVT) or pulmonary embolism (PE) When the intermitted pneumatic compression devices are activated, they force the blood out of the pressurized area allowing it to flow back in the veins. The intermitted, sequential action if the device ensures the circulation of venous blood: When the device inflates, pressure is applied in the area, squeezing the blood from the underlying deep veins forcing it to be displaced, and when the device deflates, the veins replenish with blood.

Compression devices to aid in the treatment and prevention of venous disease are not designed to be mobile; they are being bulky, heavy and restricting movement, requiring the patient to wear them while sitting or lying down. They are not designed to be customized for each individual, and expensive to be promoted for widespread personal at-home or everyday use. On the contrary, by utilizing the pneumatic soft structures one can imagine a sleeve, jacket or full lightweight flexible bodysuit that an individual can wear at any time of the day, during everyday activities to promote healthy blood circulation.

Athletes also greatly benefit from haptic interventions that improve blood circulation. Increased blood circulation allows more oxygen to flow to the muscles during physical exercise leading to less fatigue and a more efficient workout. Enhanced blood circulation also aids in muscle recovery and rebuilding. Apart from the sport massage practices, techniques for blocking and controlling blood flow in certain body parts have also been incorporated in current rehabilitation and performance enhancement strategies, such as the Blood Flow Restriction (BFR) training. The principle of BFR training lies in the fact lies in the fact that the brain falsely receives signals that the muscles of a restricted body part (i.e. a limb) during exercise are performing a more strenuous action than they do, triggering a physiological response for muscle growth and repair/regeneration.

Special sportwear made from pneumatic soft structures can aid in quick muscle recovery and rehabilitation by mimicking sports massage techniques and/or following the principles of BFR training through programmed localized pressure. Such sportswear can offer real time advanced performance if worn during high intensity exercise.

### 3. Wholistic Treatment

The emotional and physical effects of the rhythmic haptic action of the sleeve (or other garment with haptic action) should not be regarded as unrelated. In fact, the rate of respiration can affect hemodynamics. Studies have showed that controlled slow breathing can increase the heart rate and venous return. When venous return increases, the blood is being circulated around the body more efficiently. In addition, when venous return increases, muscles also increase in energy as the blood get re-oxygenated. Research shows that while cardiac activity can influence respiratory activity, the reverse influence is stronger. Because of the impact of respiration to the cardiovascular system therapeutic techniques based on breathing have been developed to aid in the treatment of circulatory diseases.

Given the dual benefits of breathing—promoting relaxation and circulatory health—one can imagine a holistic treatment based on carefully programmed haptic cycles of a pneumatic sleeve, garment or body-suit that can simultaneously promote enhanced blood circulation and lymphatic system health, full muscle energy for optimized athletic performance and rehabilitation, and emotional health by regulating stress levels.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween. It is also anticipated that steps of methods may be performed in an order different from that illustrated and still be within the meaning of the claims that follow.

### Combination of Features

The features here described can be combined in several ways. Among combinations of these features anticipated by the inventors are:

A pneumatic chamber designated (A) a textile sheet having a thermoplastic coating on an interior of the chamber. The left and right-side area of the chamber are folded inward, towards each other, in a V shape, with the point of the V directed to the interior of the chamber. The textile sheet is heat sealed at ends of the sheet to render the chamber airtight, forming a 4-layer structure in at least lateral portions of each of a first and second end of the chamber with on the left side of the chamber, in order, a top portion of the textile sheet, an upper portion of the V shape, a lower sheet portion of the V shape, and a bottom portion of the textile sheet and with on the right side of the chamber, in order, a top portion of the textile sheet, an upper portion of the V shape, a lower sheet portion of the V shape, and a bottom portion of the textile sheet. The upper portion of the V shape being sealed to the top portion of the textile sheet in a plurality of locations along the chamber and the bottom portion of the textile sheet being sealed to the bottom portion of the textile sheet in a plurality of locations along the chamber.

A pneumatic chamber designated (AA) including the pneumatic chamber designated (A) the seal of the top portion of the textile sheet in a plurality of locations along the chamber and the bottom of the textile sheet to the bottom portion of the textile sheet comprises glue.

A pneumatic chamber designated (AB) including the pneumatic chamber designated (A) and (AA) the seal of the top portion of the textile sheet in a plurality of locations along the chamber and the bottom of the textile sheet to the bottom portion of the textile sheet is comprises a heat seal.

A pneumatic chamber designated (AC) including the pneumatic chamber designated (A)-(AB) in a first portion of the chamber, the plurality of locations along the chamber where the upper portion of the sheet is heat sealed to the upper portion of the V shape is more extensive than the plurality of locations where the lower portion of the sheet is heat sealed to the lower portion of the V shape such that upon inflation the chamber curves.

A pneumatic chamber designated (AD) including the pneumatic chamber designated (A)-(AC) in a second portion of the chamber, the plurality of locations along the chamber where the lower portion of the sheet is heat sealed to the lower portion of the V shape is more extensive than the plurality of locations where the upper portion of the sheet is heat sealed to the upper portion of the V shape such that upon inflation the chamber curves in an opposite direction than curve of the chamber.

A pneumatic chamber designated (AE) including the pneumatic chamber designated (A)-(AD) the textile sheet comprises a woven nylon fabric coated with thermoplastic polyurethane.

A pneumatic chamber designated (AF) including the pneumatic chamber designated (A)-(AE) a sleeve structure configured to enclose a limb comprising a plurality of the pneumatic chambers each of the pneumatic chambers surrounding the limb, the pneumatic chambers disposed in a determined order along a length of the sleeve structure.

A pneumatic chamber designated (AG) including the pneumatic chamber designated (A)-(AF) further including a processor with memory containing a haptic program. The processor is coupled to control inflation and deflation of the pneumatic chambers with air from at least one air pump. At least one sensor configured to sense a physiological parameter selected from the group consisting of breathing rate, heart rate, skin conductance. The haptic program configured to control the inflation and deflation of the pneumatic chambers according to the sensed physiological parameter.

A pneumatic chamber designated (AH) including the pneumatic chamber designated (A)-(AF) further including a digital radio coupled to the processor and configured to link to and communicate with a base station radio.

A pneumatic chamber designated (AI) including the pneumatic chamber designated (A)-(AH), further including a touchpad. The digital radio configured to link to and communicate with the base station radio to a second system comprising a touchpad. The haptic program further configured to control the inflation and deflation of the pneumatic chambers according to input on the touchpad of the second system, and to transmit commands entered on the touchpad to the second system.

A pneumatic chamber designated (AJ) including the pneumatic chamber designated (A)-(AH) further including the second system comprising at least one sensor configured to sense a physiological parameter selected from the group consisting of breathing rate, heart rate, and skin conductance. The haptic program configured to control the inflation and deflation of the pneumatic chambers according to the sensor configured to sense a physiological parameter of the second system, and to transmit commands entered on the touchpad to the second system.

A pneumatic chamber designated (AK) a garment comprising the sleeve structure of claim (AJ), the garment selected from the group consisting of a jacket, sweater, shirt, hat, scarf, gloves, and pants.

A method designated (B) includes fitting an individual with at least one sleeve structure surrounding at least one body part of the individual, the sleeve structure comprising a plurality of pneumatic chambers, each of the pneumatic chambers wrapped around the body part, the pneumatic chambers disposed in a first determined order along a length of the sleeve structure. The method further includes coupling the pneumatic chambers to an actuator unit, the actuator unit operable under control of a processor and comprising at least one air pump, the actuator unit configured to inflate and deflate the pneumatic chambers in a determined sequence. The method further includes detecting a physiological pattern with sensors. The method further includes inflating and deflating, in the determined sequence, the plurality of pneumatic chambers disposed along the length of the sleeve structure.

A method designated (BA) including the method designated (B) the physiological patterns are selected from the group consisting of breathing rate patterns, heart rate patterns, and skin conductance patterns of the individual.

A method designated (BB) including the methods designated (B) and (BA) further including receiving, by a digital radio coupled to the processor, input, from a remotely located companion wearable device comprising a second plurality of pneumatic chambers. Each of the pneumatic chambers wrapped around a body part of a second individual. The pneumatic chambers disposed in a second determined order along a length of the companion wearable device. The method further includes inflating and deflating the plurality of pneumatic chambers disposed along the length of the sleeve structure based on the received input.

A method designated (BC) including the methods designated (B)-(BB) further including transmitting, by the digital radio, to a digital radio associated with the companion wearable device, input to inflate and deflate the second plurality of pneumatic chambers of the companion wearable device.

A method designated (BD) including the methods designated (B)-(BC) further including coupling electric resistive heat pads embedded within corresponding pneumatic chambers to a corresponding switch of a series of switches and the actuator unit, the actuator unit further configured to heat the electric resistive heat pads. The method further including heating the electric resistive heat pads.

A method designated (BE) including the methods designated (B)-(BD) further including receiving, by a digital radio coupled to the processor, input, from a remotely located control console operated by an authorized user. The method further including inflating and deflating the plurality of pneumatic chambers disposed along the length of the sleeve structure according to the received input.

A method designated (BF) including the methods designated (B)-(BE) further including inflating and deflating, in a second determined sequence, the plurality of pneumatic chambers disposed along the length of the sleeve structure according to input received by a touchpad coupled to the actuator.

A method designated (BG) including the methods designated (B)-(BF) the one sleeve structure is a garment selected from the group consisting of a jacket, sweater, shirt, hat, scarf, gloves, and pants.

1. A pneumatic chamber comprising:
a textile sheet having a thermoplastic coating on an interior of the chamber, left and right-side area of the chamber are folded inward, towards each other, in a V shape, with the point of the V directed to the interior of the chamber;
the textile sheet being heat sealed at ends of the sheet to render the chamber airtight, forming a 4-layer structure in at least lateral portions of each of a first and second end of the chamber with on the left side of the chamber, in order, a top portion of the textile sheet, an upper portion of the V shape, a lower sheet portion of the V shape, and a bottom portion of the textile sheet and with on the right side of the chamber, in order, a top portion of the textile sheet, an upper portion of the V shape, a lower sheet portion of the V shape, and a bottom portion of the textile sheet; and
the upper portion of the V shape being sealed to the top portion of the textile sheet in a plurality of locations along the chamber and the bottom portion of the textile sheet being sealed to the bottom portion of the textile sheet in a plurality of locations along the chamber.

2. The pneumatic chamber of claim 1, wherein the seal of the top portion of the textile sheet in a plurality of locations along the chamber and the bottom of the textile sheet to the bottom portion of the textile sheet comprises glue.

3. The pneumatic chamber of claim 1, wherein the seal of the top portion of the textile sheet in a plurality of locations along the chamber and the bottom of the textile sheet to the bottom portion of the textile sheet is comprises a heat seal.

4. The pneumatic chamber of claim 3, wherein in a first portion of the chamber, the plurality of locations along the chamber where the upper portion of the sheet is heat sealed to the upper portion of the V shape is more extensive than the plurality of locations where the lower portion of the sheet is heat sealed to the lower portion of the V shape such that upon inflation the chamber curves.

5. The pneumatic chamber of claim 3, wherein in a second portion of the chamber, the plurality of locations along the chamber where the lower portion of the sheet is heat sealed to the lower portion of the V shape is more extensive than the plurality of locations where the upper portion of the sheet is heat sealed to the upper portion of the V shape such that upon inflation the chamber curves in an opposite direction than curve of the chamber.

6. The pneumatic chamber of claim 1, wherein the textile sheet comprises a woven nylon fabric coated with thermoplastic polyurethane.

7. A sleeve structure configured to enclose a limb comprising a plurality of the pneumatic chambers of claim 1, each of the pneumatic chambers surrounding the limb, the pneumatic chambers disposed in a determined order along a length of the sleeve structure.

8. The sleeve structure of claim 7, further comprising:
a processor with memory containing a haptic program;
the processor coupled to control inflation and deflation of the pneumatic chambers with air from at least one air pump;
at least one sensor configured to sense a physiological parameter selected from the group consisting of breathing rate, heart rate, and skin conductance; and
the haptic program configured to control the inflation and deflation of the pneumatic chambers according to the sensed physiological parameter.

9. The sleeve structure of claim 8, further comprising a digital radio coupled to the processor and configured to link to and communicate with a base station radio.

10. The sleeve structure of claim 9, further comprising:
a touchpad;
the digital radio configured to link to and communicate with the base station radio to a second system comprising a second touchpad;
the haptic program further configured to control inflation and deflation of the pneumatic chambers according to input on a second touchpad of the second system, and to transmit commands entered on the second touchpad to the second system.

11. The sleeve structure of claim 10, further comprising:
the second system comprising at least one sensor configured to sense a physiological parameter selected from the group consisting of breathing rate, heart rate, and skin conductance;
the haptic program configured to control the inflation and deflation of the pneumatic chambers according to the sensor configured to sense a physiological parameter of the second system, and to transmit commands entered on the touchpad to the second system.

12. A garment comprising the sleeve structure of claim 11, the garment selected from the group consisting of a jacket, sweater, shirt, hat, scarf, gloves, and pants.

13. A method, comprising:
fitting an individual with at least one sleeve structure surrounding at least one body part of the individual, the sleeve structure comprising a plurality of pneumatic chambers, each of the pneumatic chambers wrapped around the body part, the pneumatic chambers disposed in a first determined order along a length of the sleeve structure;
coupling the pneumatic chambers to an actuator unit, the actuator unit operable under control of a processor and comprising at least one air pump, the actuator unit configured to inflate and deflate the pneumatic chambers in a determined sequence;
detecting, with sensors, a physiological pattern; and
inflating and deflating, in the determined sequence, the plurality of pneumatic chambers disposed along the length of the sleeve structure.

14. The method of claim 13, wherein the physiological pattern is selected from the group consisting of breathing rate patterns, heart rate patterns, and skin conductance of the individual.

15. The method of claim 13, further comprising:
receiving, by a digital radio coupled to the processor, input, from a remotely located companion wearable device comprising a second plurality of pneumatic chambers, each of the pneumatic chambers wrapped around a body part of a second individual, the pneumatic chambers disposed in a second determined order along a length of the companion wearable device; and
inflating and deflating the plurality of pneumatic chambers disposed along the length of the sleeve structure based on the received input.

16. The method of claim 15, further comprising:
transmitting, by the digital radio, to a digital radio associated with the companion wearable device, input to inflate and deflate the second plurality of pneumatic chambers of the companion wearable device.

17. The method of claim 13, further comprising:
coupling electric resistive heat pads embedded within corresponding pneumatic chambers to a corresponding switch of a series of switches and the actuator unit, the actuator unit further configured to heat the electric resistive heat pads; and
heating the electric resistive heat pads.

18. The method of claim 13, further comprising:
receiving, by a digital radio coupled to the processor, input, from a remotely located control console operated by an authorized user; and
inflating and deflating the plurality of pneumatic chambers disposed along the length of the sleeve structure according to the received input.

19. The method of claim 13, further comprising:
inflating and deflating, in a second determined sequence, the plurality of pneumatic chambers disposed along the length of the sleeve structure according to input received by a touchpad coupled to the actuator unit.

20. The method of claim 13 wherein the one sleeve structure is a garment selected from the group consisting of a jacket, sweater, shirt, hat, scarf, gloves, and pants.
